(19) United States
(12) Patent Application Publication
(10) Pub. No.: US 2012/0253206 A1
(43) Pub. Date: Oct. 4, 2012

(54) MEASUREMENT APPARATUS, MEASUREMENT METHOD, INFORMATION PROCESSING APPARATUS, INFORMATION PROCESSING METHOD, AND PROGRAM

(76) Inventors: Shinichi FUKUDA, Kanagawa (JP); Takayuki OGISO, Tokyo (JP); Hideyuki KOGURE, Chiba (JP); Hiroaki NAKANO, Tokyo (JP); Hirotaka MURAMATSU, Tokyo (JP); Akira ENDO, Saitama (JP); Hiroyuki INO, Tokyo (JP)

(21) Appl. No.: 13/427,316

(22) Filed: Mar. 22, 2012

(30) Foreign Application Priority Data
Mar. 30, 2011 (JP) 2011-076189

(51) Int. Cl. A61B 5/0205; A61B 5/053; A61B 5/0402

(52) U.S. Cl. 600/483

(57) ABSTRACT

A measurement apparatus includes a signal generation unit generating a measurement signal for measuring a bioelectrical impedance, a first electrode pair making contact with the left and right sides of a body of a person under measurement to supply the measurement signal generated to the body, a second electrode pair placed adjacent to the first electrode pair and making contact with the left and right sides of the body, a bioelectrical impedance measurement unit measuring the bioelectrical impedance of the person under measurement based on an electrical signal obtained from the second electrode pair in response to supplying of the measurement signal, and an electrocardiogram signal measurement unit measuring an electrocardiogram signal of the person under measurement based on the electrical signal obtained from the second electrode pair. The bioelectrical impedance measurement unit and the electrocardiogram signal measurement unit concurrently operate in parallel.

### BACKGROUND

The present disclosure relates to a measurement apparatus, measurement method, information processing apparatus, information processing method, and program and more particularly to a measurement apparatus, measurement method, information processing apparatus, information processing method, and program that can accurately detect a heartbeat pattern indicating the motion of a human heart.

Previously, electrocardiogram signals have been measured for medical purposes such as check-up examination. Electrocardiogram signals are electrical signals caused by the cyclic motion of a human heart and the characteristic of the waveform pattern (referred to below as heartbeat pattern) of one cycle varies between individuals.

FIG. 1 shows the waveform of a general heartbeat pattern. In FIG. 1, the horizontal axis represents the time axis (sample axis) and the vertical axis represents the electric potential. As shown in FIG. 1, characteristic waves including a U wave, P wave, Q wave, R wave, S wave, and T wave in this order are arranged in a general heartbeat pattern.

A proposal has been made to use this heartbeat pattern for individual authentication (for example, in Japanese Unexamined Patent Application Publication (Translation of PCT Application) No. 2008-518709). Specifically, the electrocardiogram signals of registrants are measured, the heartbeat patterns are extracted, and their feature quantities are calculated and registered in advance. During authentication, the electrocardiogram signal of a person under authentication is measured, the heartbeat pattern is extracted, the feature quantity is calculated and compared with the registered feature quantities, and authentication is made on the basis of the comparison results.

A general method used in medical institutions that make highly accurate measurements is the 12-lead system in which electrodes are attached to 12 points on the head, chest, four limbs, etc. to measure electrocardiogram signals. As shown in FIG. 2, there has been a simpler method (referred to below as the simple measurement method) in which a left-hand electrode L, a right-hand electrode R, and a ground electrode G, which is attached to the left foot etc. are used for measurement.

Since the voltage of a human body should be identical to the reference potential of the electrocardiogram signal measurement unit for measurement of the electrocardiogram signal, the human body and the electrocardiogram signal measurement unit are grounded. However, since the difference in voltage between the human body and the electrocardiogram signal measurement unit becomes zero over time even when only the left-hand electrode L and the right-hand electrode R are used, the electrocardiogram signal can be measured. For more immediate and accurate measurements, however, it is preferable to use the ground electrode G in addition to the left-hand electrode L and the right-hand electrode R.

There has been a method similar to the simple measurement method for electrocardiogram signals, in which the body impedance (also referred to below as a bioelectrical Z) is measured on the basis of an electric signal flowing between electrodes, or through the human body. The bioelectrical Z is measured in the state where, for example, a person under measurement brings his or her left hand into contact with two electrodes L1 and L2 and his or her right hand into contact with two electrodes R1 and R2, as shown in FIG. 3. The person under measurement may bring his or her bottoms of both feet instead of both hands into contact with the electrodes.

Specifically, as shown in FIG. 4, an alternate current i with a frequency of tens of kilohertz is fed between the electrode L1 and the electrode R1 as a bioelectrical Z measurement signal, the potential difference V_{z} between the electrode L2 and the electrode R2 is measured, and the bioelectrical Z is calculated on the basis of the expression V_{z}=i·Z. The bioelectrical Z measured in this way is converted into body composition data (percent of body fat, muscle amount, bone amount) using tables and functions retained in advance, and given to the person under measurement.

### SUMMARY

As described above, there have been the method of measuring an electrocardiogram signal and the method of measuring a bioelectrical Z, but these methods are carried out by different apparatuses, so the electrocardiogram signal and bioelectrical Z are not measured concurrently.

It is desirable to measure the electrocardiogram signal and bioelectrical Z concurrently.

According to an embodiment of the present disclosure, there is provided a measurement apparatus including a signal generation unit that generates a measurement signal for measuring bioelectrical impedance, a first electrode pair that makes contact with a left side and a right side of a body of a person under measurement to supply the measurement signal generated to the body of the person under measurement, a second electrode pair that is placed adjacent to the first electrode pair and makes contact with the left side and the right side of the body of the person under measurement, a bioelectrical impedance measurement unit that measures the bioelectrical impedance of the person under measurement based on an electrical signal obtained from the second electrode pair in response to supplying of the measurement signal, and an electrocardiogram signal measurement unit that measures an electrocardiogram signal of the person under measurement based on the electrical signal obtained from the second electrode pair, in which the bioelectrical impedance measurement unit and the electrocardiogram signal measurement unit concurrently operate in parallel.

The measurement apparatus according to the embodiment of the present disclosure may further include an adjustment unit that makes an average potential of the body of the person under measurement with which the first electrode pair makes contact identical to a reference potential of the electrocardiogram signal measurement unit.

The adjustment unit may be a current amplifier disposed between a power supply unit and the first electrode pair, one of a positive input terminal and a negative input terminal included in the current amplifier being grounded.

The electrocardiogram signal measurement unit may include a filter unit that extracts a frequency component corresponding to the electrocardiogram signal from the electrical signal obtained from the second electrode pair.

The bioelectrical impedance measurement unit may detect a voltage difference of the electrical signal obtained from the second electrode pair in response to supplying of the measurement signal and may calculate the bioelectrical impedance of the person under measurement based on a detection signal indicating the voltage difference detected and a current of the measurement signal.

The bioelectrical impedance measurement unit may include a filter unit that extracts the same frequency component as in the measurement signal from the detection signal.

The measurement apparatus according to the embodiment of the present disclosure may further include an extraction unit that extracts a heartbeat pattern indicating cyclic motion of a heart from the electrocardiogram signal measured, in which the extraction unit may restrict the extraction of the heartbeat pattern based on the bioelectrical impedance measured.

According to the embodiment of the present disclosure, there is provided a measurement method carried out by a measurement apparatus measuring a bioelectrical impedance and an electrocardiogram signal of a person under measurement, the method including, generating a measurement signal for measuring the bioelectrical impedance, supplying the measurement signal generated to a body of the person under measurement from a first electrode pair in contact with left and right sides of the body, measuring the bioelectrical impedance of the person under measurement using a second electrode pair placed adjacent to the first electrode pair based on an electrical signal obtained in response to supplying of the measurement signal, the second electrode pair being in contact with the left and right sides of the body, and measuring an electrocardiogram signal of the person under measurement based on the electrical signal obtained from the second electrode pair, in which the bioelectrical impedance and the electrocardiogram signal are measured concurrently in parallel.

According to the embodiment of the present disclosure, there is provided a program that lets a computer execute a process including, generating a measurement signal for measuring a bioelectrical impedance, supplying the measurement signal generated to a body of a person under measurement from a first electrode pair in contact with left and right sides of the body, measuring the bioelectrical impedance of the person under measurement using a second electrode pair placed adjacent to the first electrode pair based on an electrical signal obtained in response to supplying of the measurement signal, the second electrode pair being in contact with the left and right sides of the body, and measuring an electrocardiogram signal of the person under measurement based on the electrical signal obtained from the second electrode pair, in which the bioelectrical impedance and the electrocardiogram signal are measured concurrently in parallel.

In the embodiment of the present disclosure, the measurement signal for measuring bioelectrical impedance is generated and the measurement signal generated is supplied to the body of the person under measurement from the first electrode pair in contact with the left and right sides of the person under measurement. Then, the bioelectrical impedance of the person under measurement is measured using the second electrode pair that is placed adjacent to the first electrode pair and in contact with the left and right sides of the body of the person under measurement, based on an electrical signal obtained in response to supplying of the measurement signal. Concurrently with this, the electrocardiogram signal of the person under measurement is measured in parallel based on the electrical signal obtained from the second electrode pair.

According to another embodiment of the present disclosure, there is provided an information processing apparatus including a bioelectrical impedance measurement unit that measures a bioelectrical impedance of a person under measurement, an electrocardiogram signal measurement unit that measures an electrocardiogram signal of the person under measurement concurrently with the measurement of the bioelectrical impedance, an extraction unit that extracts a heartbeat pattern indicating cyclic motion of a heart from the electrocardiogram signal measured, and a processing unit that performs predetermined processing using the heartbeat pattern extracted, in which the extraction unit restricts the extraction of the heartbeat pattern based on the bioelectrical impedance measured.

The extraction unit may extract the heartbeat pattern when the bioelectrical impedance measured is equal to or less than a first threshold or may stop extracting the heartbeat pattern when the bioelectrical impedance measured is more than the first threshold.

The processing unit may perform authentication by registering the heartbeat pattern corresponding to the person under measurement assumed as a registrant and comparing the heartbeat pattern corresponding to the person under measurement assumed as a person under authentication with the heartbeat pattern(s) of the registered registrant(s).

The processing unit may perform authentication by registering the heartbeat pattern and the bioelectrical impedance corresponding to the person under measurement assumed as a registrant and comparing a correlation coefficient indicating a correlation between the heartbeat pattern corresponding to the person under measurement assumed as a person under authentication and the heartbeat pattern registered of the registrant with a second threshold that depends on a difference in the bioelectrical impedance between the registrant and the person under authentication.

According to the other embodiment of the present disclosure, there is provided an information processing method carried out by an information processing apparatus, the method including measuring a bioelectrical impedance and an electrocardiogram signal of a person under measurement concurrently, extracting a heartbeat pattern indicating cyclic motion of a heart from the electrocardiogram signal measured while making restrictions based on the bioelectrical impedance measured, and performing predetermined processing using the heartbeat pattern extracted.

According to the other embodiment of the present disclosure, there is provided a program letting a computer execute a process including measuring a bioelectrical impedance and an electrocardiogram signal of a person under measurement concurrently, extracting a heartbeat pattern indicating cyclic motion of a heart from the electrocardiogram signal measured while making restrictions based on the bioelectrical impedance measured, and performing predetermined processing using the heartbeat pattern extracted.

According to the other embodiment of the present disclosure, the bioelectrical impedance and the electrocardiogram signal of a person under measurement are measured concurrently, a heartbeat pattern indicating the cyclic motion of a heart is extracted from the electrocardiogram signal measured while making restrictions based on the bioelectrical impedance measured, and predetermined processing is performed using the heartbeat pattern extracted.

According to the embodiment of the present disclosure, the electrocardiogram signal and the bioelectrical Z can be measured concurrently.

According to the other embodiment of the present disclosure, the electrocardiogram signal and the bioelectrical Z can be measured concurrently and a good heartbeat pattern can be obtained from the electrocardiogram signal.

### BRIEF DESCRIPTION OF THE DRAWINGS

FIG. 1 shows the waveform of a general heartbeat pattern.

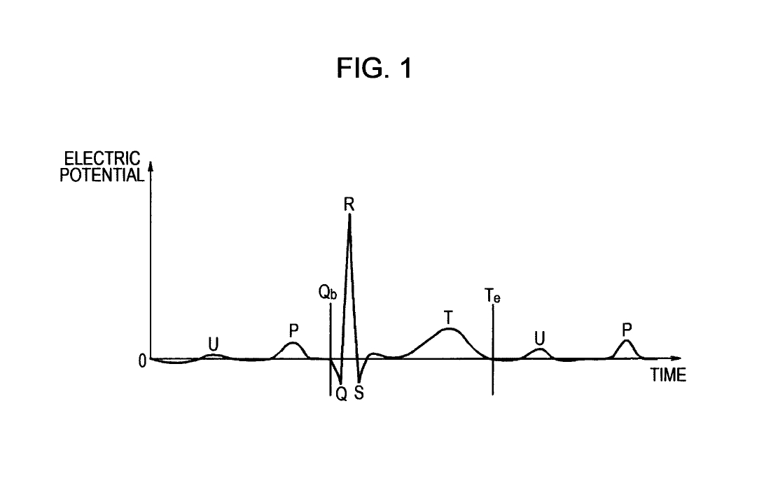

FIG. 2 shows a method of measuring an electrocardiogram signal using three electrodes.

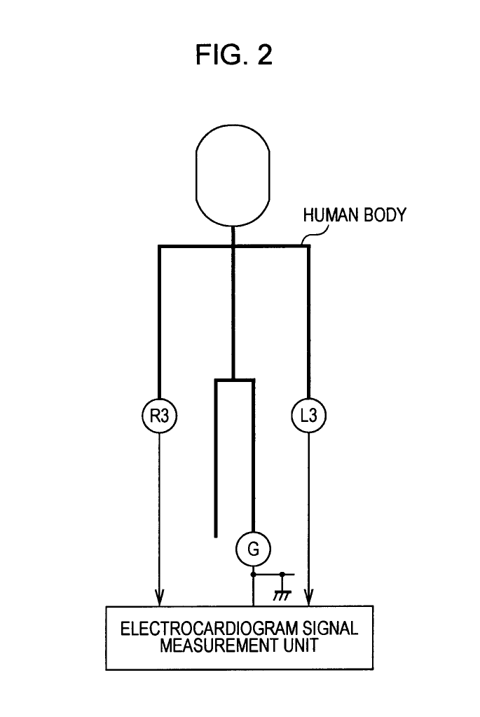

FIG. 3 shows a method of measuring a bioelectrical Z.

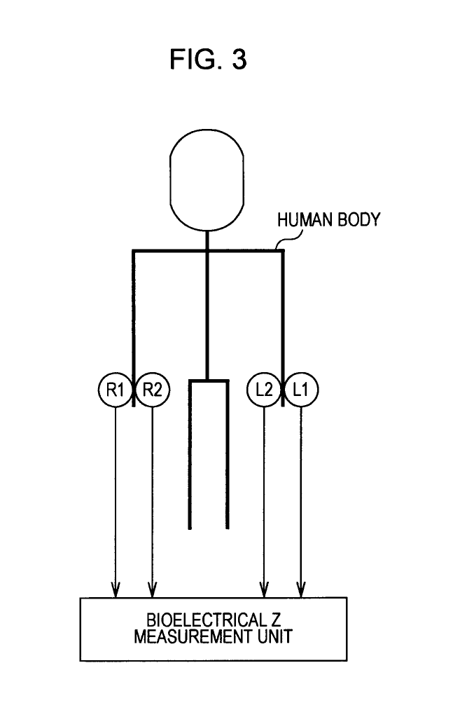

FIG. 4 shows the method of measuring a bioelectrical Z.

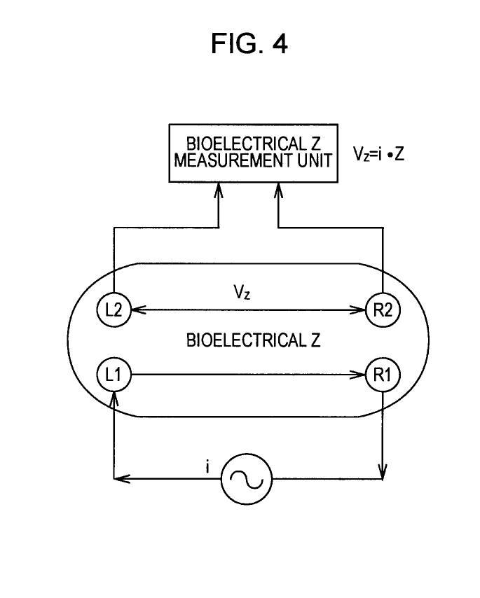

FIGS. 5A and 5B are outline views showing a measurement apparatus as an embodiment.

FIG. 6 is a block diagram showing an example of the structure of the measurement apparatus.

FIG. 7 shows a left-hand inner electrode and a right-hand inner electrode that function as ground electrodes.

FIG. 8 describes that the electrocardiogram signal can be measured even if electrocardiogram signal measurement electrodes are adjacent to ground electrodes.

FIG. 9 is a flowchart showing concurrent measurement performed by the measurement apparatus.

FIG. 10 shows an example of the waveforms of an electrocardiogram signal and a bioelectrical Z measured concurrently.

FIG. 11 is a view in which the waveform of the electrocardiogram signal in FIG. 10 is enlarged horizontally.

FIGS. 12A and 12B are outline views showing an authentication apparatus as another embodiment.

FIG. 13 is a block diagram showing an example of the structure of the authentication apparatus.

FIG. 14 is a flowchart describing the registration by the authentication apparatus.

FIG. 15 is a flowchart describing the authentication by the authentication apparatus.

FIG. 16 shows how to use modifications of the measurement apparatus and the authentication apparatus.

FIGS. 17A and 17B show a first modification.

FIGS. 18A and 18B show a second modification.

FIGS. 19A and 19B show a third modification.

FIG. 20 is a block diagram showing an example of the structure of a computer.

### DETAILED DESCRIPTION OF EMBODIMENTS

Preferred embodiments (referred to below as embodiments) of the present disclosure will be described in detail with reference to the drawings.

### 1. Embodiment

### [Example of the Structure of a Measurement Apparatus]

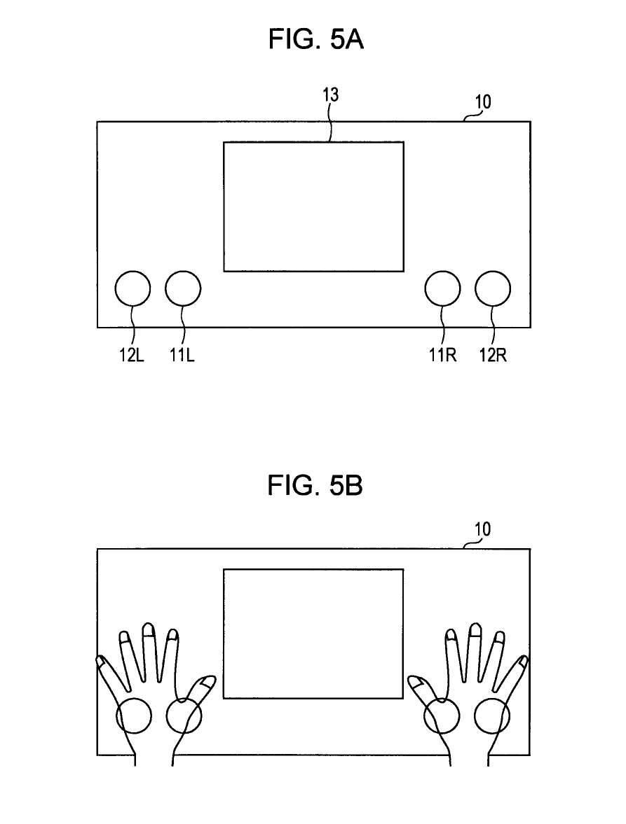

FIGS. 5A and 5B outline the upper surface of a measurement apparatus as an embodiment. The measurement apparatus 10 measures the electrocardiogram signal and the bioelectrical Z of a person under measurement concurrently.

As shown in FIG. 5A, a left-hand inner electrode 11L and a left-hand outer electrode 12L are disposed in the left of the measurement apparatus 10 and a right-hand inner electrode 11R and a right-hand outer electrode 12R are disposed in the right. In addition, an indication unit 13 is disposed at the center of the upper surface. The indication unit 13 shows the person under measurement the waveform of the electrocardiogram signal resulting from measurement and body composition values (such as the percentage of body fat) based on the bioelectrical Z.

As shown in FIG. 5B, the measurement apparatus 10 makes measurements in the state where the person under measurement brings his or her left palm into contact with the left-hand inner electrode 11L and the left-hand outer electrode 12L and his or her right palm into contact with the right-hand inner electrode 11R and the right-hand outer electrode 12R.

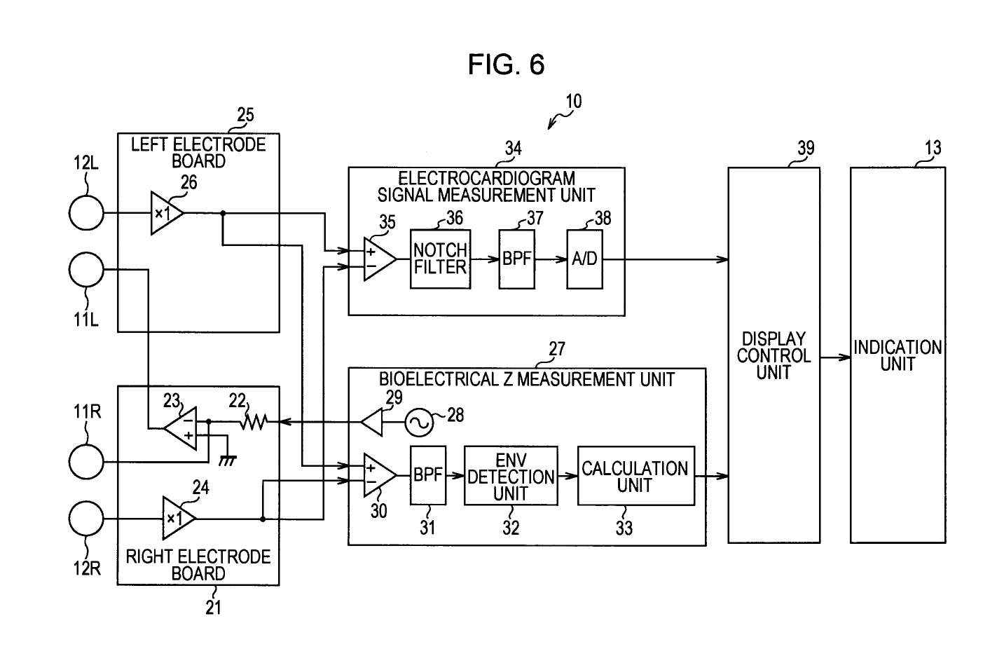

FIG. 6 shows an example of the structure of the measurement apparatus 10. The measurement apparatus 10 includes a right electrode board 21 to which the right-hand inner electrode 11R and the right-hand outer electrode 12R are connected, a left electrode board 25 to which the left-hand inner electrode 11L and the left-hand outer electrode 12L are connected, a bioelectrical Z measurement unit 27, an electrocardiogram signal measurement unit 34, a display control unit 39, and the indication unit 13.

The right electrode board 21 includes a resistor 22, a current amplifier 23, and a buffer amplifier 24. The resistor 22 is connected in series between a negative input terminal of the current amplifier 23 and a signal generation unit 28 of the bioelectrical Z measurement unit 27. The resistance of the resistor 22 is, for example, 1 kΩ. The negative input terminal of the current amplifier 23 is connected to the signal generation unit 28 of the bioelectrical Z measurement unit 27 and the right-hand inner electrode 11R. An output terminal of the current amplifier 23 is connected to the left-hand inner electrode 11L via the left electrode board 25. The positive input terminal of the current amplifier 23 is grounded. The current amplifier 23 amplifies a bioelectrical Z measurement signal i (with a frequency of 50 kHz and a voltage of 1 V, for example) input from the negative input terminal to 1 mA and output the signal to the left-hand inner electrode 11L.

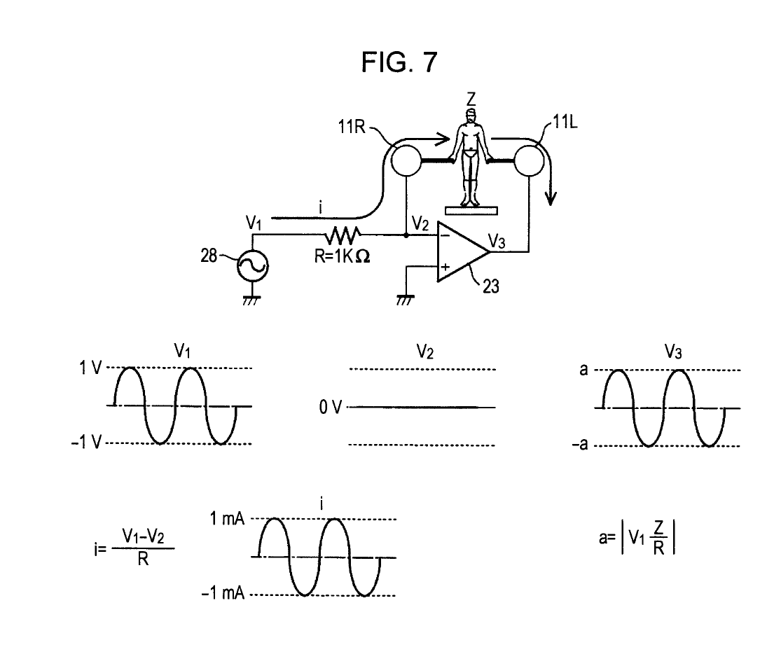

Accordingly, the amplified bioelectrical Z measurement signal i flows through a route including the left-hand inner electrode 11L, the inside of the body (living body) of the person under measurement, and the right-hand inner electrode 11R (of course, the signal flows through the route reversely). When the current amplifier 23 functions normally, the electric potential of the positive input terminal equals that of the negative input terminal. Since the positive input terminal of the current amplifier 23 is grounded, however, the electric potential of the negative input terminal also becomes 0 V. In addition, the average electric potential of an output/input terminal of the current amplifier 23 also becomes 0 V. Accordingly, the left-hand inner electrode 11L and the right-hand inner electrode 11R operate as ground electrodes of the person under measurement. Details will be given later with reference to FIG. 7.

The buffer amplifier 24 amplifies an electrical signal input from the right-hand outer electrode 12R and outputs it to the subsequent state. This electrical signal is split into two and output to the negative input terminal of an amplifier 30 in the bioelectrical Z measurement unit 27 and the negative input terminal of an amplifier 35 in the electrocardiogram signal measurement unit 34.

The left electrode board 25 has a buffer amplifier 26. The buffer amplifier 26 amplifies an electrical signal input from the left-hand outer electrode 12L and outputs it to the subsequent stage. This electrical signal is split into two and output to the positive input terminal of the amplifier 30 in the bioelectrical Z measurement unit 27 and the positive input terminal of the amplifier 35 in the electrocardiogram signal measurement unit 34.

The bioelectrical Z measurement unit 27 includes the signal generation unit 28, an amplifier 29, the amplifier 30, a BPF 31, an ENV detection unit 32, and a calculation unit 33. The signal generation unit 28 generates the bioelectrical Z measurement signal i. The amplifier 29 amplifies the bioelectrical Z measurement signal i and outputs it to the right electrode board 21.

The amplifier 30 amplifies an electrical signals input from the left-hand outer electrode 12L and the right-hand outer electrode 12R and outputs them to the BPF 31. The BPF 31 passes only the same frequency band (50 kHz) as in the bioelectrical Z measurement signal i among the electrical signal from the amplifier 30, toward the ENV detection unit 32 in the subsequent stage. The ENV detection unit 32 detects the envelope of the electrical signal input from the BPF 31 and outputs it to the calculation unit 33. The calculation unit 33 obtains a differential voltage V_{Z} between the left-hand outer electrode 12L and the right-hand outer electrode 12R from the envelope detected by the ENV detection unit 32 and calculates the bioelectrical Z (=V_{Z}/i) from the differential voltage V_{Z} and the bioelectrical Z measurement signal i. The calculated bioelectrical Z is output to the display control unit 39 in the subsequent state.

The electrocardiogram signal measurement unit 34 includes the amplifier 35, a notch filter 36, a BPF 37, and an A/D converter 38.

The amplifier 35 amplifies the electrical signals input from the left-hand outer electrode 12L and the right-hand outer electrode 12R using 0 V as the reference voltage and outputs them to the notch filter 36. The notch filter 36 and the BPF 37 extract only the frequency components of up to 100 Hz, which are major components of the electrocardiogram signal, from electrical signals output from the amplifier 30 and output them to the A/D converter 38. The A/D converter 38 digitizes the electrical signals of up to 100 Hz from the BPF 37 to generate an electrocardiogram signal. The generated electrocardiogram signal is output to the display control unit 39 in the subsequent stage.

The display control unit 39 converts the bioelectrical Z input from the bioelectrical Z measurement unit 27 into body composition values (such as the percentage of body fat) using tables and functions incorporated in advance, generates the display data, and outputs it to the indication unit 13. Based on the electrocardiogram signal input from the electrocardiogram signal measurement unit 34, the display control unit 39 also generates the display data and output it to the indication unit 13.

The indication unit 13 provides body composition values and the waveform of an electrocardiogram signal for the person under measurement based on the display data from the display control unit 39. The indication unit 13 also displays a message that instructs the person under measurement to make contact with the electrodes or retry contact with the electrodes or a message that reports measurement error.

### [Description of the Reason Why the Left-Hand Inner Electrode 11L and the Right-Hand Inner Electrode 11R Become Ground Electrodes]

FIG. 7, which indicates the peripheral circuit of the current amplifier 23, describes that the left-hand inner electrode 11L and the right-hand inner electrode 11R become ground electrodes.

As described above, the bioelectrical Z measurement signal i with a frequency of 50 kHz, a voltage V₁ of 1 V, and a current of 1 mA flows through the route including the left-hand inner electrode 11L, the human body, and the right-hand inner electrode 11R. The electric potential V₂ of the negative input terminal of the current amplifier 23 is 0 V and the average of the electric potential V₃ of the input/output terminal of the current amplifier 23 is also 0 V. The electric potential of the person under measurement in contact with the left-hand inner electrode 11L and the right-hand inner electrode 11R also becomes 0 V. Accordingly, the left-hand inner electrode 11L and the right-hand inner electrode 11R are assumed to function as ground electrodes of the person under measurement.

### [Description of the Reason Why the Electrocardiogram Signal can be Measured Even When the Electrocardiogram Signal Measurement Electrodes are Adjacent to the Ground Electrodes]

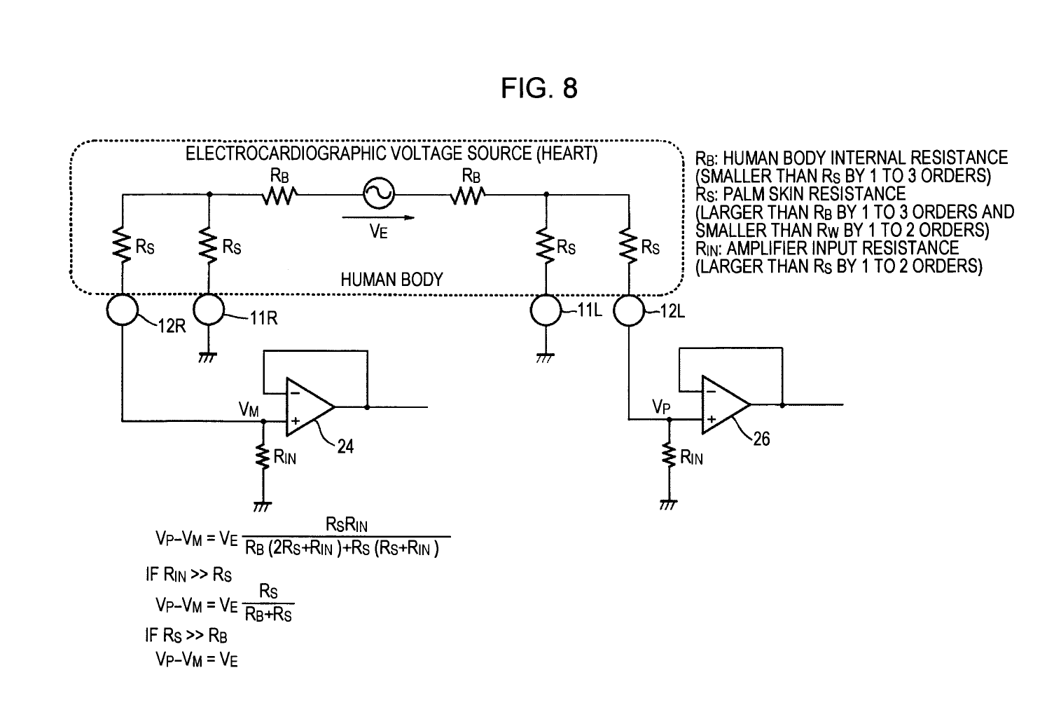

FIG. 8 describes that the electrocardiogram signal can be measured even when the electrocardiogram signal measurement electrodes (left-hand outer electrode 12L and right-hand outer electrode 12R) are adjacent to the ground electrodes (left-hand inner electrode 11L and the right-hand inner electrode 11R).

Here, the bioelectrical Z is separated into a body resistance R_{B} and a palm skin resistance R_{S}. In addition, it is assumed that the internal resistance of the buffer amplifiers 24 and 26, which amplify the electrical signals from the left-hand outer electrode 12L and right-hand outer electrode 12R is R_{IN}.

As compared with the palm skin resistance R_{S}, the body resistance R_{B} is sufficiently small because the human body mainly contains liquid and the internal resistance R_{IN} is sufficiently large. In this case, an electrocardiographic voltage V_{E} caused by the motion of a heart is measured as the differential voltage (V_{P}−V_{M}) between the buffer amplifier 24 and the buffer amplifier 26, regardless of the distance between the left-hand outer electrode 12L and the left-hand inner electrode 11L (or between the right-hand outer electrode 12R and the right-hand inner electrode 11R).

### [Operation of Measurement Apparatus 10]

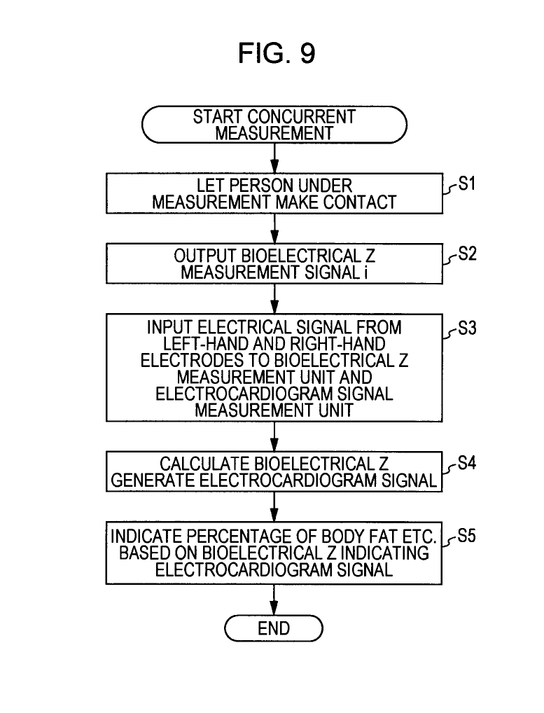

FIG. 9 is a flowchart describing processing (referred to below as a concurrent measurement) in which the measurement apparatus 10 measures a bioelectrical Z and an electrocardiogram signal.

In step S1, the person under measurement is prompted to make contact with electrodes. In response to this, the person under measurement brings his or her left palm into contact with the left-hand inner electrode 11L and the left-hand outer electrode 12L and his or her right palm into contact with the right-hand inner electrode 11R and the right-hand outer electrode 12R.

In step S2, the signal generation unit 28 of the bioelectrical Z measurement unit 27 starts outputting the bioelectrical Z measurement signal i. The bioelectrical Z measurement signal i flows through the route including the left-hand inner electrode 11L, the human body, and the right-hand inner electrode 11R.

In step S3, an electrical signal from the left-hand outer electrode 12L is input to the bioelectrical Z measurement unit 27 and the electrocardiogram signal measurement unit 34. In step S4, the bioelectrical Z measurement unit 27 calculates a bioelectrical Z and outputs it to the display control unit 39. At the same time with this, the electrocardiogram signal measurement unit 34 generates an electrocardiogram signal and outputs it to the display control unit 39.

In step S5, the display control unit 39 converts the calculated bioelectrical Z into body composition values (such as the percentage of body fat) using tables and functions retained in advance, generates the display data, and outputs it to the indication unit 13. Based on the electrocardiogram signal input from the electrocardiogram signal measurement unit 34, the display control unit 39 also generates the display data and outputs it to the indication unit 13. The indication unit 13 provides body composition values and the waveform of an electrocardiogram signal for the person under measurement based on the display data from the display control unit 39. Now, the concurrent measurement is completed.

In the concurrent measurement described above, the bioelectrical Z and the electrocardiogram signal can be measured concurrently without being time-divided. Since the bioelectrical Z and electrocardiogram signal can be measured concurrently, predetermined processing (such as authentication described later) that uses the bioelectrical Z and electrocardiogram signal can be performed quickly.

### 2. Another Embodiment

First, the relationship between the bioelectrical Z and the electrocardiogram signal will be described. Then, an authentication apparatus as another embodiment that uses the bioelectrical Z and electrocardiogram signal for personal authentication will be described.

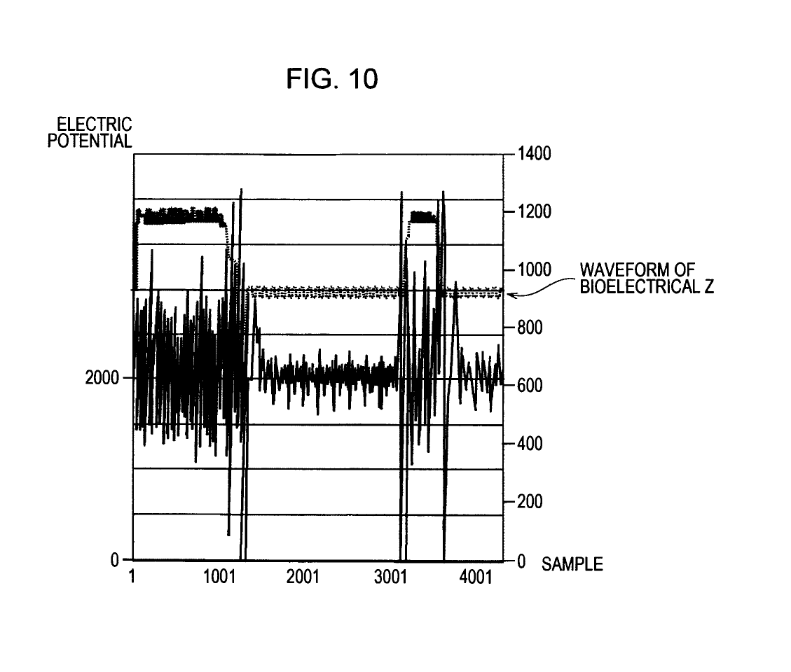

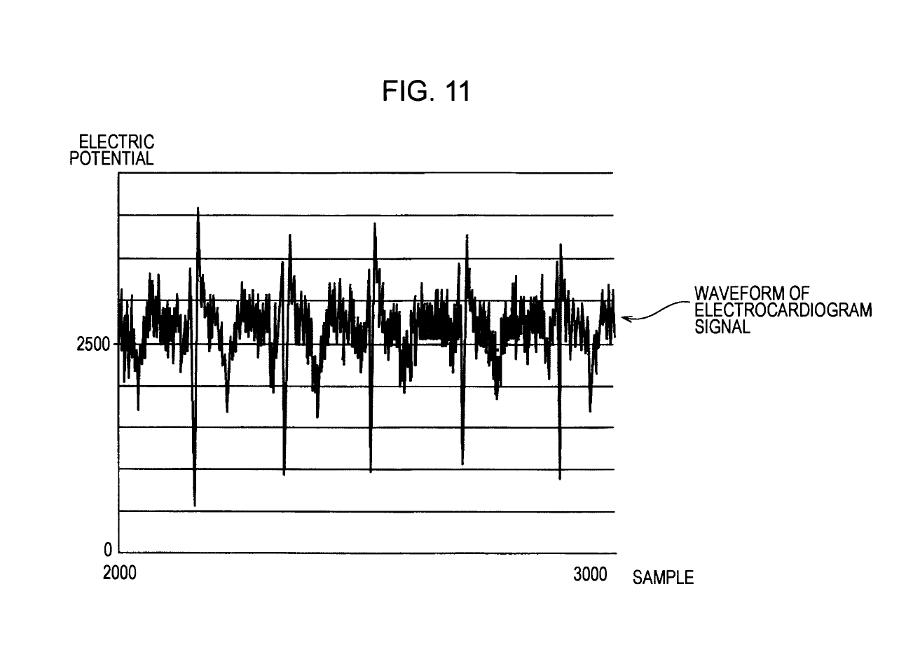

FIG. 10 shows an example of the waveforms of the bioelectrical Z and electrocardiogram signal measured concurrently. In FIG. 10, the sample number is plotted on the horizontal axis and the electric potential is plotted on the vertical axis. FIG. 11 is an enlarged view of the electrocardiogram signal in FIG. 10 in the range from sample numbers 2000 to 3000.

It is found that the electrocardiogram signal shown in FIGS. 10 and 11 has a stable waveform in the range from sample numbers 2000 to 3000. It is also found that the electrocardiogram signal has unstable waveforms due to inclusion of noise components in the other ranges. Inclusion of noise components is caused by, for example, loose connection between the palm and electrodes, variations in the muscle potential of the living body, etc.

It is found that the bioelectrical Z in FIG. 10 indicates low values in the range from sample numbers 2000 to 3000 and the range equal to or more than 3500; the bioelectrical Z indicates high values in the other ranges. As shown in FIG. 10, there is a correlation between the electrocardiogram signal and the bioelectrical Z; the bioelectrical Z becomes high when the waveform of the electrocardiogram signal is unstable and the bioelectrical Z becomes low when the waveform of the electrocardiogram signal is stable.

In the registration and authentication described later, (the feature quantity of) a heartbeat pattern extracted from the electrocardiogram signal is associated with the person under measurement (registrant or person under authentication). To improve the accuracy of personal authentication, the heartbeat pattern should be extracted from the stable electrocardiogram signal.

Accordingly, the authentication apparatus as the other embodiment references the bioelectrical Z and extracts the heartbeat pattern only from the electrocardiogram signal when the bioelectrical Z is equal to or less than a predetermined value.

### [Example of the Structure of Authentication Apparatus]

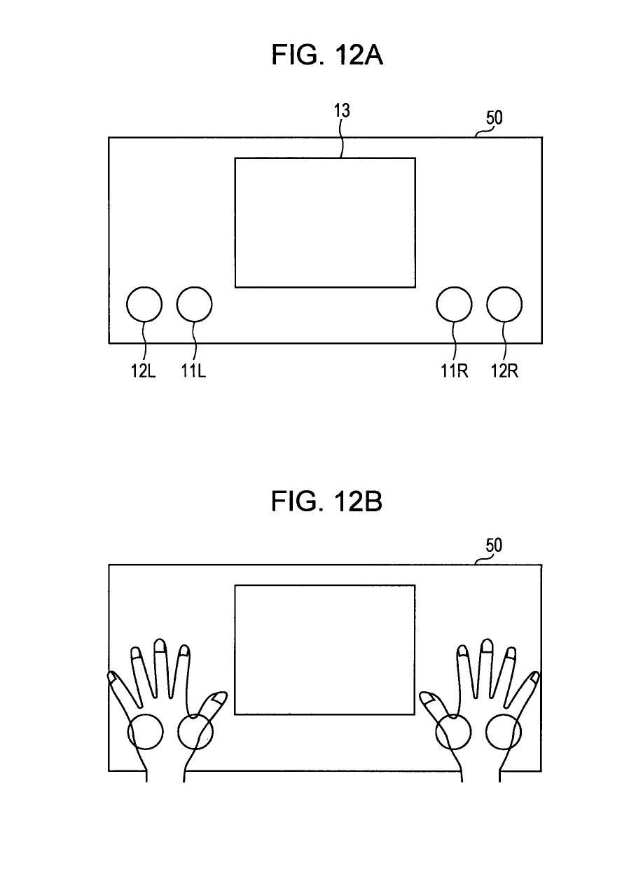

FIGS. 12A and 12B are outline views showing the upper surface of the authentication apparatus as the other embodiment. This authentication apparatus 50 measures the electrocardiogram signal and the bioelectrical Z of the person under measurement (registrant or person under authentication) concurrently and performs personal authentication using a heartbeat pattern extracted from the electrocardiogram signal.

Of the components of the authentication apparatus 50, those common to the authentication apparatus 10 as the embodiment are given the same reference numerals and their descriptions are omitted as appropriate.

As shown in FIG. 12A, the left-hand inner electrode 11L and the left-hand outer electrode 12L are disposed in the left of the authentication apparatus 50; the right-hand inner electrode 11R and the right-hand outer electrode 12R are disposed in the right. In addition, an indication unit 13, which shows measurement results, authentication results, etc., is disposed at the center of the upper surface.

As shown in FIG. 12B, the authentication apparatus 50 makes measurements in the state where the person under measurement brings his or her left palm into contact with the left-hand inner electrode 11L and the left-hand outer electrode 12L and his or her right palm into contact with the right-hand inner electrode 11R and the right-hand outer electrode 12R and then performs personal authentication.

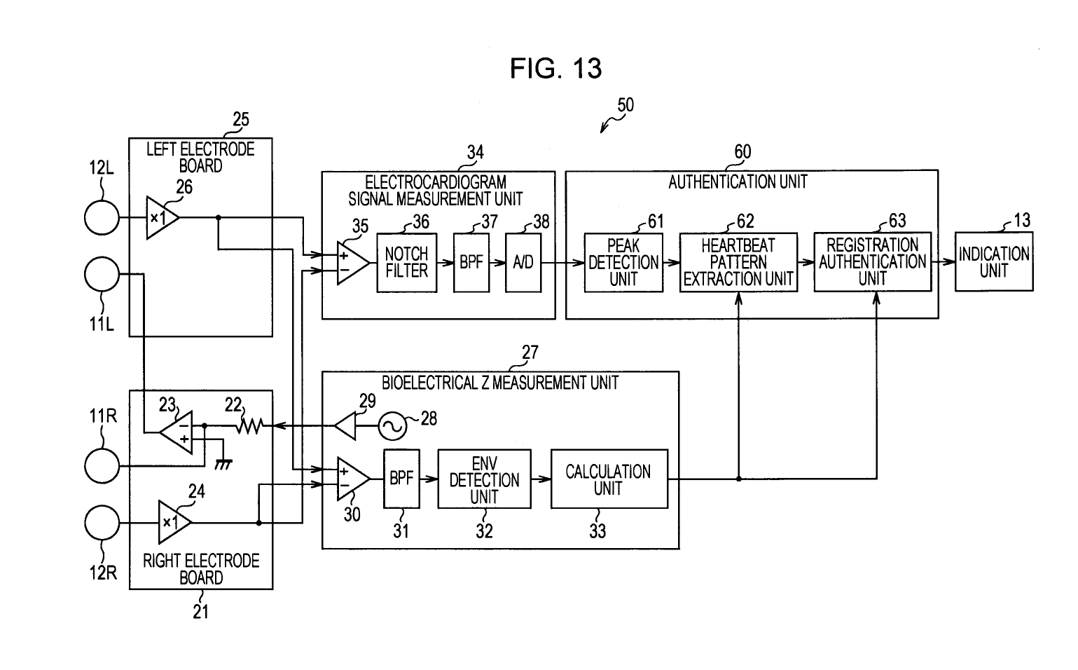

FIG. 13 shows an example of the structure of the authentication apparatus 50. The authentication apparatus 50 includes a right electrode board 21 to which the right-hand inner electrode 11R and the right-hand outer electrode 12R are connected and a left electrode board 25 to which the left-hand inner electrode 11L and the left-hand outer electrode 12L are connected, a bioelectrical Z measurement unit 27, an electrocardiogram signal measurement unit 34, an authentication unit 60, and the indication unit 13.

The bioelectrical Z measurement unit 27 reports the calculated bioelectrical Z to a heartbeat pattern extraction unit 62 of the authentication unit 60 and a registration authentication unit 63. The electrocardiogram signal measurement unit 34 outputs the generated electrocardiogram signal to a peak detection unit 61 of the authentication unit 60.

The authentication unit 60 includes the peak detection unit 61, the heartbeat pattern extraction unit 62, and the registration authentication unit 63.

The peak detection unit 61 detects the peak of a characteristic wave (for example, an R wave) in the electrocardiogram signal and reports it to the heartbeat pattern extraction unit 62. Only when the bioelectrical Z is equal to or less than a predetermined first threshold, the heartbeat pattern extraction unit 62 extracts a predetermined sample range relative to the detected peak from the electrocardiogram signal as a heartbeat pattern, calculates its feature quantity, and outputs it to the registration authentication unit 63. The method of calculating the feature quantity of a heartbeat pattern is arbitrary. The heartbeat pattern itself may be assumed to be the feature quantity.

During registration, the registration authentication unit 63 associates a person (registrant) under measurement with the feature quantity of a heartbeat pattern and the bioelectrical Z measured when the heartbeat pattern is extracted and records (registers) it. During authentication, the registration authentication unit 63 calculates a correlation value indicating the correlation between the feature quantity of the heartbeat pattern of the person under measurement (person under authentication) and the feature quantity of each of registered heartbeat patterns and performs the personal authentication of the person under authentication based on the correlation value.

Specifically, the registration authentication unit 63 identifies the feature quantity with the highest correlation value among the feature quantities of the heartbeat patterns of registered registrants and, when the correlation value is equal to or more than the predetermined second threshold, authenticates the person under authentication as the corresponding registrant.

The predetermined second threshold may be a fixed value or may be a variable value that depends on the difference between the bioelectrical Z of the person under authentication and the bioelectrical Z of the registrant to be compared. For the same person, the bioelectrical Z varies with the measurement timing, but the variation is small. Accordingly, as the difference between the bioelectrical Z of the person under authentication and the bioelectrical Z of the registrant with the highest correlation becomes larger, the second threshold should be larger.

When it is assumed that the correlation value ranges from −1 to 1 and the highest correlation value is 1, if, for example, the difference between the bioelectrical Z of the person under authentication and the bioelectrical Z of the registrant may be 170Ω or less, the second threshold is set to 0.99; if the difference is 170 to 340Ω, the second threshold may be 0.995; if the difference is 340Ω or more, the second threshold may be 0.999.

The registration authentication unit 63 outputs the result of personal authentication to the indication unit 13. In addition, when the bioelectrical Z is more than the first threshold, the registration authentication unit 63 lets the indication unit 13 display a message indicating measurement error etc.

The indication unit 13 displays the result of personal authentication input from the registration authentication unit 63. In addition, the indication unit 13 displays a message that instructs the person under measurement to make contact with the electrodes or retry contact with the electrodes or a message that reports measurement error, under control of the registration authentication unit 63.

### [Operation of Authentication Apparatus 50]

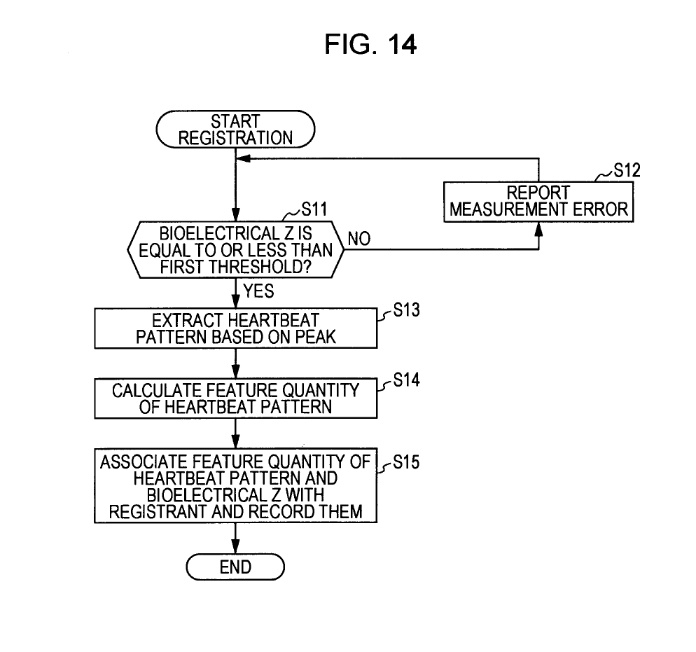

FIG. 14 is a flowchart describing the registration by the authentication apparatus 50.

The registration assumes that the bioelectrical Z and electrocardiogram signal concurrently measured from the registrant have been input to the authentication unit 60 through processing similar to the concurrent measurement by the measurement apparatus 10. It is also assumed that the peak detection unit 61 has detected the peak of the electrocardiogram signal input from the previous stage.

In step S11, the heartbeat pattern extraction unit 62 and the registration authentication unit 63 determine whether the bioelectrical Z is equal to or less than the first threshold. When the bioelectrical Z is determined to be more than the first threshold, since the waveform of the electrocardiogram signal measured at this time is thought to be unstable, the processing proceeds to step S12. In step S12, the indication unit 13 displays a message indicating measurement error etc. under control of the registration authentication unit 63. In response to this message, the registrant takes an action such as retrying contact with electrodes.

When the bioelectrical Z is determined to be equal to or less than the first threshold in step S11, since the waveform of the electrocardiogram signal measured at this time is thought to be stable, the processing proceeds to step S13. The heartbeat pattern extraction unit 62 extracts a predetermined sample range relative to the detected peak from the electrocardiogram signal as a heartbeat pattern in step S13, and calculates its feature quantity and outputs it to the registration authentication unit 63 in step S14.

In step S15, the registration authentication unit 63 associates the person (registrant) under measurement with the feature quantity of the heartbeat pattern and the bioelectrical Z measured when the heartbeat pattern is extracted and records (registers) it. Now, the registration is completed.

In the above registration, the heartbeat pattern is not extracted when the electrocardiogram signal is thought to be stable and the heartbeat pattern is extracted only when the electrocardiogram signal is thought to be stable. Accordingly, (the feature quantity of) the reliable heartbeat pattern corresponding to the registrant can be registered.

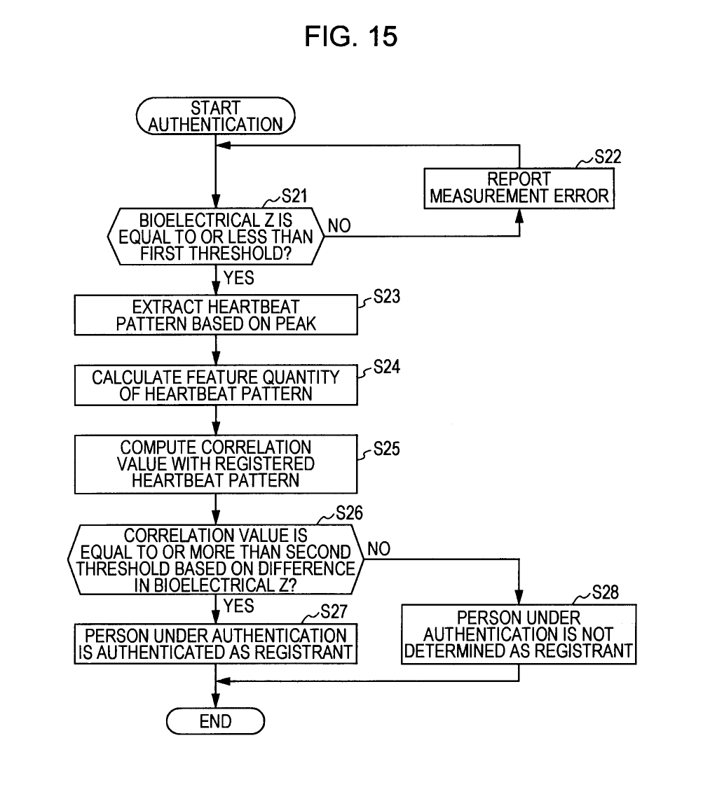

FIG. 15 is a flowchart describing the authentication by the authentication apparatus 50.

The authentication assumes that the bioelectrical Z and electrocardiogram signal concurrently measured from the person under authentication have been input to the authentication unit 60 through processing similar to the concurrent measurement by the measurement apparatus 10. It is also assumed that the peak detection unit 61 has detected the peak of the electrocardiogram signal input from the previous stage.

In step S21, the heartbeat pattern extraction unit 62 and the registration authentication unit 63 determine whether the bioelectrical Z is equal to or less than the first threshold. When the bioelectrical Z is determined to be more than the first threshold, since the waveform of the electrocardiogram signal measured at this time is thought to be unstable, the processing proceeds to step S22. In step S22, the indication unit 13 displays a message indicating measurement error etc. under control of the registration authentication unit 63. In response to this message, the person under authentication takes an action such as retrying contact with electrodes.

On the other hand, when the bioelectrical Z is determined to be equal to or less than the first threshold, since the waveform of the electrocardiogram signal measured at this time is thought to be stable, the processing proceeds to step S23. The heartbeat pattern extraction unit 62 extracts a predetermined sample range relative to the detected peak from the electrocardiogram signal as a heartbeat pattern in step S23, and calculates its feature quantity and outputs it to the registration authentication unit 63 in step S24.

In step S25, the registration authentication unit 63 calculates the correlation value between the feature quantity of the heartbeat pattern of the person under authentication and the feature quantities of the registered heartbeat patterns. In step S26, the registration authentication unit 63 identifies the registrant with the highest correlation value as a result of the calculation and determines whether the highest correlation value is equal to or more than the second threshold that depends on the difference between the bioelectrical Z of the identified registrant and the bioelectrical Z of the person under authentication.

When the highest correlation value is determined to be equal to or more than the second threshold, the processing proceeds to step S27. In step S27, the registration authentication unit 63 notifies the indication unit 13 that the person under authentication is authenticated as the registrant. The indication unit 13 notifies the person under authentication that the person under authentication is authenticated as the registrant.

On the other hand, when the highest correlation value is determined to be less than the second threshold, the processing proceeds to step S28. In step S28, the registration authentication unit 63 notifies the indication unit 13 that there is no registrant who matches the person under authentication. The indication unit 13 notifies the person under authentication that there is no registrant who matches the person under authentication. Now, the authentication is completed.

In the above registration, the heartbeat pattern is not extracted when the electrocardiogram signal is thought to be stable and the heartbeat pattern is extracted only when the electrocardiogram signal is thought to be stable. Accordingly, (the feature quantity of) the reliable heartbeat pattern corresponding to the person under authentication can be registered, thereby improving the accuracy of authentication.

### 3. Modifications

Next, modifications of the measurement apparatus 10 as the embodiment and the authentication apparatus 50 as the other embodiment will be described.

The positions of the four electrodes of the measurement apparatus 10 (authentication apparatus 50) may be changed as described below.

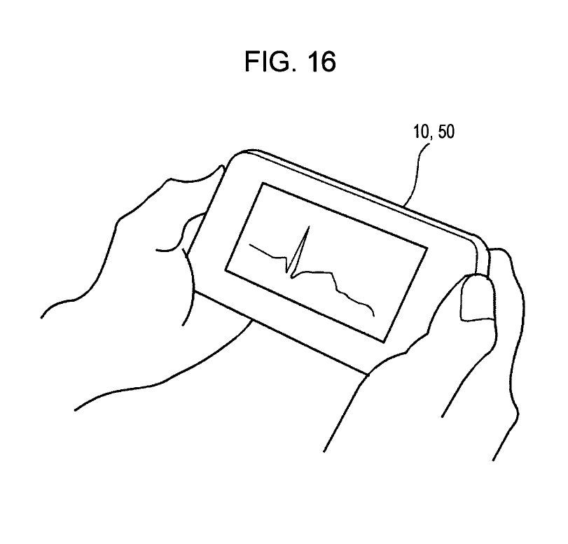

FIG. 16 shows how to use the measurement apparatus 10 (authentication apparatus 50) for which the positions of the four electrodes have been changed. That is, the four electrodes may be arranged so that two electrode make contact with each of left and right palms or fingers in the state where the person under measurement holds the measurement apparatus 10 (authentication apparatus 50) with two hands.

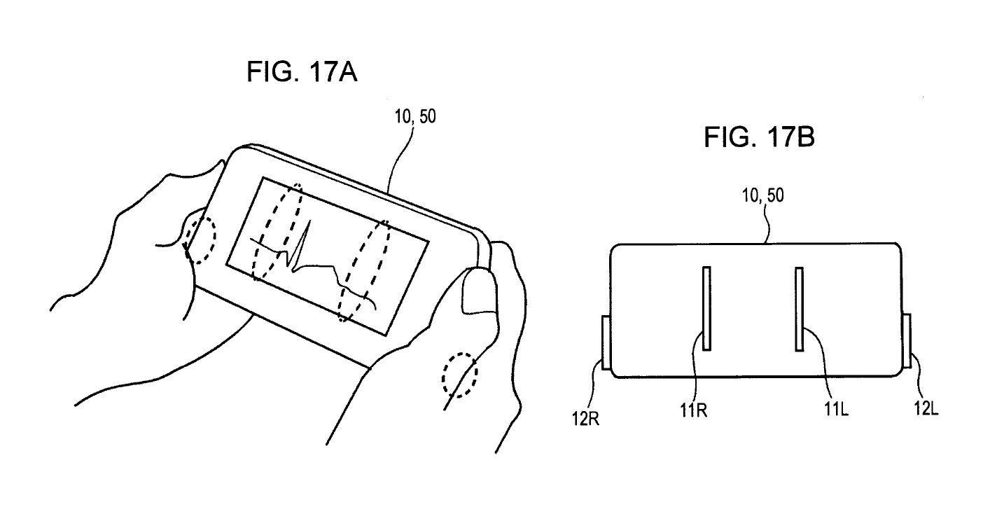

FIGS. 17A and 17B show a modification in which a left-hand outer electrode 12L is arranged on the left side of the body of the measurement apparatus 10 (authentication apparatus 50), a right-hand outer electrode 12R is arranged on the right side, and a left-hand inner electrode 11L and a right-hand inner electrode 11R is arranged near the center of the back of the body.

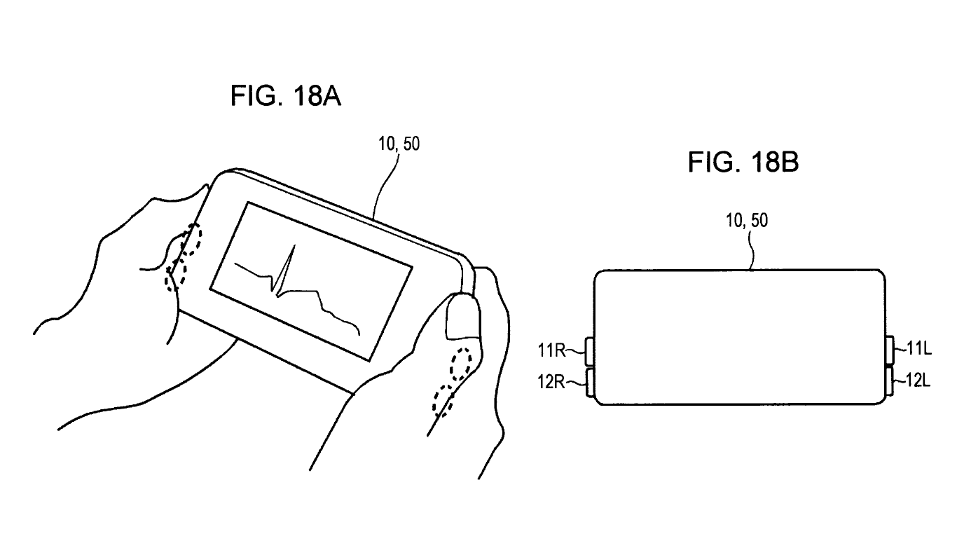

FIGS. 18A and 18B show a modification in which the left-hand inner electrode 11L and the left-hand outer electrode 12L are arranged on the left side of the body of the measurement apparatus 10 (authentication apparatus 50) and the right-hand inner electrode 11R and the right-hand outer electrode 12R are arranged on the right side.

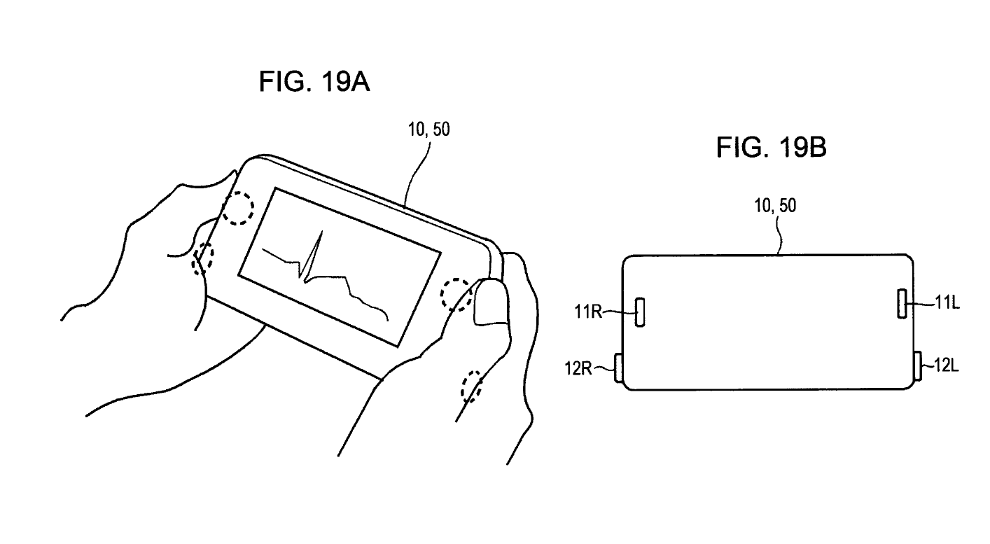

FIGS. 19A and 19B show a modification in which the left-hand outer electrode 12L is arranged on the left side of the body of the measurement apparatus 10 (authentication apparatus 50), the right-hand outer electrode 12R is arranged on the right side, the left-hand inner electrode 11L is arranged in the left on the back of the body, and the right-hand inner electrode 11R is arranged in the right on the back of the body.

The four electrodes can be arranged in a way other than in the modifications shown in FIGS. 17A to 19B.

The series of processes described above may be implemented through hardware or software. When the series of processes is implemented through software, a computer that has dedicated hardware incorporating the programs constituting the software is used or the programs are installed from a program storage medium in, for example, a general-purpose personal computer, which executes various functions according to programs installed.

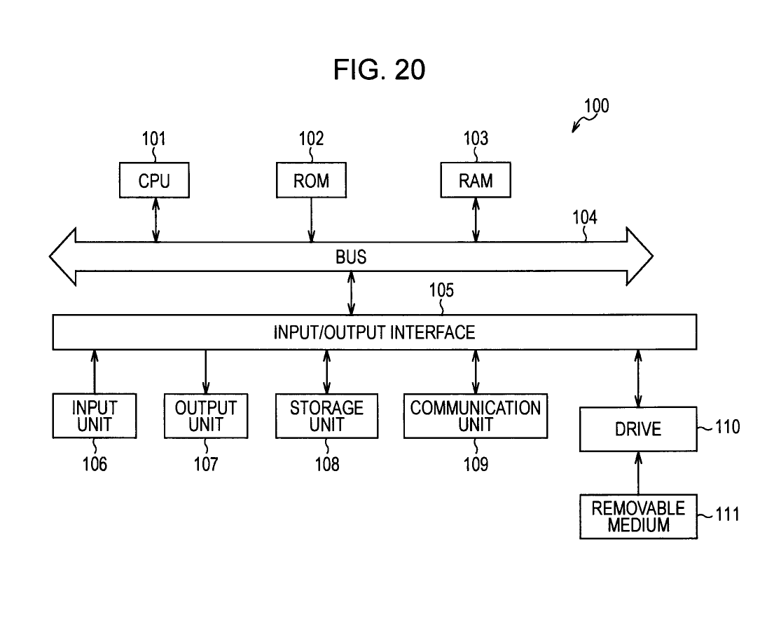

FIG. 20 is a block diagram showing an example of the hardware structure of a computer that uses programs to execute the series of processes described above.

In the computer 100, a CPU (central processing unit) 101, a ROM (read only memory) 102, and a RAM (random access memory) 103 are interconnected through a bus 104.

An input/output interface 105 is also connected to the bus 104. A input unit 106 including a keyboard, mouse, microphone, etc., a output unit 107 including a display, speaker, etc., a storage unit 108 including a hard disk drive, non-volatile memory, etc., a communication unit 109 including a network interface etc., and a drive 110 driving a removal medium 111 such as a magnetic disc, optical disc, magnetic optical disc, or semiconductor memory are connected to the input/output interface 105.

In the computer 100 configured as described above, the CPU 101 loads in the RAM 103 a program stored in the storage unit 108 via the input/output interface 105 and the bus 104 and executes the program to perform the series of processes described above.

The program executed by the computer may be a program in which processes are carried out in chronological sequence according to the order described in this specification or a program in which processes are carried out in parallel or at a necessary timing such as an occurrence of a call.

The program may be processed by one computer or may be processed by a plurality of computers in a distributed manner. In addition, the program may be transferred to a remote computer for execution.

In this specification, the system represents a whole apparatus including a plurality of units.

Embodiments of the present disclosure are not limited to the above embodiments and various modifications may be made without departing from the scope of the present disclosure.

The present disclosure contains subject matter related to that disclosed in Japanese Priority Patent Application JP 2011-076189 filed in the Japan Patent Office on Mar. 30, 2011, the entire contents of which are hereby incorporated by reference.

What is claimed is:

1. A measurement apparatus comprising:
a signal generation unit that generates a measurement signal for measuring a bioelectrical impedance;
a first electrode pair that makes contact with a left side and a right side of a body of a person under measurement to supply the measurement signal generated to the body of the person under measurement;
a second electrode pair that is placed adjacent to the first electrode pair and makes contact with the left side and the right side of the body of the person under measurement;
a bioelectrical impedance measurement unit that measures the bioelectrical impedance of the person under measurement based on an electrical signal obtained from the second electrode pair in response to supplying of the measurement signal; and
an electrocardiogram signal measurement unit that measures an electrocardiogram signal of the person under measurement based on the electrical signal obtained from the second electrode pair;
wherein the bioelectrical impedance measurement unit and the electrocardiogram signal measurement unit concurrently operate in parallel.

2. The measurement apparatus according to claim 1, further comprising an adjustment unit that makes an average potential of the body of the person under measurement with which the first electrode pair makes contact identical to a reference potential of the electrocardiogram signal measurement unit.

3. The measurement apparatus according to claim 2, wherein the adjustment unit is a current amplifier disposed between a power supply unit and the first electrode pair, one of a positive input terminal and a negative input terminal included in the current amplifier being grounded.

4. The measurement apparatus according to claim 2, wherein the electrocardiogram signal measurement unit includes a filter unit that extracts a frequency component corresponding to the electrocardiogram signal from the electrical signal obtained from the second electrode pair.

5. The measurement apparatus according to claim 2, wherein the bioelectrical impedance measurement unit detects a voltage difference of the electrical signal obtained from the second electrode pair in response to supplying of the measurement signal and calculates the bioelectrical impedance of the person under measurement based on a detection signal indicating the voltage difference detected and a current of the measurement signal.

6. The measurement apparatus according to claim 5, wherein the bioelectrical impedance measurement unit includes a filter unit that extracts the same frequency component as in the measurement signal from the detection signal.

7. The measurement apparatus according to claim 2, further comprising:
an extraction unit that extracts a heartbeat pattern indicating cyclic motion of a heart from the electrocardiogram signal measured,
wherein the extraction unit restricts the extraction of the heartbeat pattern based on the bioelectrical impedance measured.

8. A measurement method carried out by a measurement apparatus measuring a bioelectrical impedance and an electrocardiogram signal of a person under measurement, the method comprising:
generating a measurement signal for measuring the bioelectrical impedance;
supplying the measurement signal generated to a body of the person under measurement from a first electrode pair in contact with left and right sides of the body;
measuring the bioelectrical impedance of the person under measurement using a second electrode pair placed adjacent to the first electrode pair based on an electrical signal obtained in response to supplying of the measurement signal, the second electrode pair being in contact with the left and right sides of the body; and
measuring an electrocardiogram signal of the person under measurement based on the electrical signal obtained from the second electrode pair;
wherein the bioelectrical impedance and the electrocardiogram signal are measured concurrently in parallel.

9. A program that lets a computer execute a process comprising:
generating a measurement signal for measuring a bioelectrical impedance;
supplying the measurement signal generated to a body of a person under measurement from a first electrode pair in contact with left and right sides of the body;
measuring the bioelectrical impedance of the person under measurement using a second electrode pair placed adjacent to the first electrode pair based on an electrical signal obtained in response to supplying of the measurement signal, the second electrode pair being in contact with the left and right sides of the body; and
measuring an electrocardiogram signal of the person under measurement based on the electrical signal obtained from the second electrode pair;
wherein the bioelectrical impedance and the electrocardiogram signal are measured concurrently in parallel.

10. An information processing apparatus comprising:
a bioelectrical impedance measurement unit that measures a bioelectrical impedance of a person under measurement;
an electrocardiogram signal measurement unit that measures an electrocardiogram signal of the person under measurement concurrently with the measurement of the bioelectrical impedance;
an extraction unit that extracts a heartbeat pattern indicating cyclic motion of a heart from the electrocardiogram signal measured; and
a processing unit that performs predetermined processing using the heartbeat pattern extracted;
wherein the extraction unit restricts the extraction of the heartbeat pattern based on the bioelectrical impedance measured.

11. The information processing apparatus according to claim 10, wherein the extraction unit extracts the heartbeat pattern when the bioelectrical impedance measured is equal to or less than a first threshold or stops extracting the heartbeat pattern when the bioelectrical impedance measured is more than the first threshold.

12. The information processing apparatus according to claim 10, wherein the processing unit performs authentication by registering the heartbeat pattern corresponding to the person under measurement assumed as a registrant and comparing the heartbeat pattern corresponding to the person under measurement assumed as a person under authentication with the heartbeat pattern registered of the registrant.

13. The information processing apparatus according to claim 12, wherein the processing unit performs authentication by registering the heartbeat pattern and the bioelectrical impedance corresponding to the person under measurement assumed as a registrant and comparing a correlation coefficient indicating a correlation between the heartbeat pattern corresponding to the person under measurement assumed as a person under authentication and the heartbeat pattern registered of the registrant with a second threshold that depends on a difference in the bioelectrical impedance between the registrant and the person under authentication.

14. An information processing method carried out by an information processing apparatus, the method comprising:
measuring a bioelectrical impedance and an electrocardiogram signal of a person under measurement concurrently;
extracting a heartbeat pattern indicating cyclic motion of a heart from the electrocardiogram signal measured while making restrictions based on the bioelectrical impedance measured; and
performing predetermined processing using the heartbeat pattern extracted.

15. A program that lets a computer execute a process comprising:
measuring a bioelectrical impedance and an electrocardiogram signal of a person under measurement concurrently;
extracting a heartbeat pattern indicating cyclic motion of a heart from the electrocardiogram signal measured while making restrictions based on the bioelectrical impedance measured; and
performing predetermined processing using the heartbeat pattern extracted.
